(19) United States
(12) Patent Application Publication
(10) Pub. No.: US 2024/0424096 A1
(43) Pub. Date: Dec. 26, 2024

(54) IL-10 Expressing Cells For Enhanced Cancer Immunotherapies

(71) Applicant: ECOLE POLYTECHNIQUE FEDERALE DE LAUSANNE (EPFL), Lausanne (CH)

(72) Inventors: Yugang GUO, Hangzhou (CN); Li TANG, Echichens (CH); Yang ZHAO, Lausanne (CH)

(21) Appl. No.: 18/684,249

(22) PCT Filed: Aug. 23, 2022
(86) PCT No.: PCT/EP2022/073462

§ 371 Date: Feb. 16, 2024

(30) Foreign Application Priority Data
Aug. 24, 2021 (EP) 21192853.6

(51) Int. Cl. A61K 39/00; A61K 38/20; A61P 35/00; C07K 14/725; C07K 16/22; C07K 16/28; C07K 16/40

(52) U.S. Cl. CPC A61K 39/4611; A61K 38/2066; A61K 39/4631; A61K 39/464404; A61K 39/464406; A61K 39/464412; A61K 39/464456; A61P 35/00; C07K 14/7051; C07K 16/22; C07K 16/2803; C07K 16/2863; C07K 16/40

(57) ABSTRACT

The present invention relates generally to the field of anti-cancer therapy, in particular to the use of adoptive T cell transfer therapy for treating cancer, in particular sold tumors. More specifically, the present invention relates to immune cells comprising one or more recombinant constructs, wherein at least one recombinant construct encodes an interleukin-10, a fragment or a variant thereof.

### FIELD OF THE INVENTION

The present invention relates generally to the field of anti-cancer therapy, in particular to the use of adoptive T cell transfer therapy for treating cancer, in particular solid tumors. More specifically, the present invention relates to immune cells comprising one or more recombinant constructs, wherein at least one recombinant construct encodes an interleukin-10, a fragment or a variant thereof.

### BACKGROUND OF THE INVENTION

Chimeric antigen receptor (CAR) T cells and T cell receptor (TCR) transgenic T cells (both called ‘adoptive T cell transfer’ therapy) are genetically engineered T-cell based adoptive transfer immunotherapies. For example, CAR T cells have shown promising results in the clinic, particularly in hematologic malignancies, but has limited progress in solid tumors (Lim, W. A. & June, C. H. The Principles of Engineering Immune Cells to Treat Cancer. Cell 168, 724-740 (2017)).

It has been reported that CAR T cells in the tumor microenvironment (TME) show the loss of effector functions and proliferative capacity, defined as T cell ‘exhaustion’, which can be raised by persistent antigen stimulation and other metabolic stress in solid tumor (Schietinger, A. et al. Tumor-Specific T Cell Dysfunction Is a Dynamic Antigen-Driven Differentiation Program Initiated Early during Tumorigenesis. Immunity 45, 389-401 (2016); Vodnala, S. K. et al. T cell stemness and dysfunction in tumors are triggered by a common mechanism. Science 363, (2019)).

It has been reported that exhausted T cells exhibit suppressed mitochondrial respiration, and such poor metabolic fitness may reinforce T cell exhaustion and impair their antitumor immune response. Metabolic intervention during the expansion phase of adoptively transferred CAR T cells has been shown to modulate in vivo differentiation and to improve antitumor response (Alizadeh, D. et al. IL15 Enhances CAR T Cell Antitumor Activity by Reducing mTORC1 Activity and Preserving Their Stem Cell Memory Phenotype. Cancer Immunol. Res. 7, 759-772 (2019)).

However, this type of intervention led to a suboptimal antitumor effect, which may be caused by either the cytokines applied are not constitutively supplied or their limited metabolic reprogramming capacity to fully rescue T cells from exhaustion.

Thus, the development of engineering processes supporting immune cell metabolic fitness, expansion, and survival within the TME, as well as effective immune cells with enhanced antitumor activity, are still urgently needed.

### SUMMARY OF THE INVENTION

The present invention provides an immune cell expressing an interleukin-10, a fragment or a variant thereof, said immune cell comprising one or more recombinant constructs, wherein at least one recombinant construct encodes an interleukin-10, a fragment or a variant thereof.

Further provided is a nucleic acid sequence encoding a nucleic acid sequence encoding one or more recombinant constructs of the invention.

Further provided is a plasmid or a vector comprising a nucleic acid sequence of the invention.

Further provided is a pharmaceutical composition comprising i) an immune cell of the invention, ii) a nucleic acid of the invention, and/or iii) a plasmid or a vector of the invention, and at least one pharmaceutically acceptable carrier or diluent.

Also provided is a method of treatment and/or prevention of a cancer comprising administering a pharmaceutical composition of the invention to a subject in need thereof.

Also provided is a method of treatment and/or prevention of a cancer in a subject comprising (i) removing and isolating immune cells, preferably native T cells, from said subject, or providing immune cells, preferably native T cells, (ii) genetically engineering said T cells with at least one recombinant construct encoding an interleukin-10, a fragment or a variant thereof and with a second recombinant construct encoding a chimeric antigen receptor (CAR), a T cell receptor (TCR) or any other synthetic tumor targeting motif or antigen, (iii) expanding ex vivo into a larger population of engineered T cells, and (iv) reintroducing into the patient or subject.

Also provided is a method of enhancing antitumor activity in a subject comprising (i) removing and isolating immune cells, preferably native T cells, from said subject, or providing immune cells, preferably native T cells, (ii) genetically engineering said T cells with at least one recombinant construct encoding an interleukin-10, a fragment or a variant thereof and with a second recombinant construct encoding a chimeric antigen receptor (CAR), a T cell receptor (TCR) or any other synthetic tumor targeting motif or antigen, (iii) expanding ex vivo into a larger population of engineered T cells, and (iv) reintroducing into the patient or subject.

### DESCRIPTION OF THE FIGURES

FIG. 1—HER2-specific CAR T cells coexpressing IL-10 (IL-10 HER2 CAR T) enhances OXPHOS of CAR T cells upon antigen stimulation and promotes CAR T cell proliferation. (a) Schematic representations of HER2-directed second generation CAR (HER2 CAR), and HER2-directed second generation CAR modified to express murine IL-10 following a 2A element (HER2 CAR-IL-10). (b) Transduction with HER2 CAR or HER2 CAR-IL-10 construct was conducted by retroviral vectors. The expression levels of CAR were analyzed by flow cytometry. The numbers in histograms represent the percentages of HER2

CAR positively stained cells. Similar results were obtained from ten independent experiments. (c) CAR T cells were co-cultured with mitomycin C-treated MC38-HER2 (HER2-expressing MC38 colon cancer cells) for 3 days. The culture supernatants were examined for concentrations of IL-10 by enzyme-linked immunosorbent assay (ELISA). (d, e) CAR T cells were labeled with cell tracker CFSE and were co-cultured with mitomycin-C-treated MC38-HER2 cells at an effector: target (E: T) ratio of 1:1 for the indicated periods. (d) Absolute numbers of viable HER2 CAR T or IL-10 HER2 CAR T on different days. (e) The percentage of HER2 CAR T or IL-10 HER2CAR T cell division in the presence of phosphate-buffered saline (PBS), isotype control antibody, or anti-IL-10 mAb. (f) Representative oxygen consumption rate (OCR) trace of CAR T cells stimulated with MC38-HER2 at the E: T ratio of 5:1 for 24 h. (g) Representative extracellular acidification rate (ECAR) trace for cells treated as (f). (h) Statistical analysis of basal OCR from (f). (i) Statistical analysis of basal ECAR from (g). (j) Ratios of OCR/ECAR from (f) and (g). Data represent the mean ±SEM. Similar results were obtained from at least three independent experiments.

FIG. 2—IL-10 HER2 CAR T cell enhances antitumor activity of CAR T cells through a pyruvate-dependent manner. (a) Cytotoxicity assay using MC38-HER2 cells as targets. Target cells were mixed with HER2 CAR T or IL-10 HER2 CAR T cells at the indicated E/T ratios. (b, c, d) Untransduced control T cell (Ctrl T), HER2 CAR T in the presence of 0 and 145 ng/ml of mouse recombinant IL-10 (mIL-10), or IL-10 HER2 CAR T cells co-culture with MC38-HER2 cells at the ET ratio of 0.5:1 for 48 h. (b) MC38-HER2 tumor cell killing percentage, (c) viable CAR T cell counts were analyzed by flow cytometry. (d) Ratio of polyfunctional CAR T cells in coculture or resting phase was assessed by intracellular cytokine staining.

FIG. 3—IL-10 HER2 CAR T therapy eradicates established mouse MC38-HER2 colon adenocarcinoma. C57BL/6 mice were inoculated subcutaneously with MC38-HER2 cells (3×10⁵) and received intravenously (i.v.) adoptive cell transfer of HER2 CAR T cells (3×10⁶), IL-10 HER2 CAR T cells (3×10⁶), or HER2 CAR T cells (3×10⁶) followed by i.v. administration of mIL-10 (1 μg) on day 6, respectively. Shown are average tumor growth curves (a) and survival curves (b) of each treatment group. Shown are numbers of long-term-surviving mice among the total number of mice in the group. (c) Survivors from treatment groups of IL-10 HER2 CAR T monotherapy were rechallenged subcutaneously with MC38-HER2 (1×10⁶) cells on day 90 post primary inoculation. Naive WT mice (n=5) were inoculated with the same number of tumor cells as controls. Shown are survival curves and numbers of long-term-surviving mice against the re-challenges. Data represent the mean ±SEM.

FIG. 4—TRP-1 IL-10 CAR T therapy prolongs survival in mouse B16F10 melanoma model. (a) Schematic representations of TRP-1-directed second generation CAR (TRP-1 CAR), and TRP-1-directed second generation CAR modified to express murine IL-10 following a 2A element (IL-10 TRP-1 CAR). (b) Transduction with TRP-1 CAR or IL-10 TRP-1 CAR construct was conducted by retroviral vectors. The expression levels of CAR were analyzed by flow cytometry. The numbers in histograms represent the percentages of c-Myc tag positively stained cells. Similar results were obtained from ten independent experiments. (c, d) TRP-1 CAR T in the presence of 0 and 145 ng/mL of mouse recombinant IL-10 (mIL-10), or IL-10 TRP-1 CAR T cells co-culture with B16F10 cells at the E:T ratio of 0.5:1 for 48 h. (c) B16F10 tumor cell killing percentage, and (d) viable CAR T cell counts were analyzed by flow cytometry. (e, f) C57BL/6 mice were inoculated subcutaneously with B16F10 melanoma cells (3×10⁵) and received i.v. adoptive cell transfers of TRP-1 CAR T cells (3×10⁶), IL-10 TRP-1 CAR T cells (3×10⁶) on day 6, respectively. Shown are average tumor growth curves (e) and survival curves (f) of each treatment group. Data represent the mean ±SEM.

FIG. 5—Complete regression of pre-established mouse 4T1-Luc-EGFRvIII metastatic mammary carcinoma model by treatment with IL-10 EGFRvIII CAR T cells. (a) Schematic representations of EGFRvIII-directed second generation CAR (EGFRvIII CAR), and EGFRvIII-directed second generation CAR modified to express murine IL-10 following a 2A element (IL-10 EGFRvIII CAR). (b) The expression levels of CAR were analyzed by flow cytometry. The numbers in histograms represent the percentages of c-Myc tag positively stained cells. Similar results were obtained from ten independent experiments. (c) CAR T cells were co-cultured with mitomycin C-treated 4T1-Luc-EGFRvIII for 3 days. The culture supernatants were examined for concentrations of IL-10 by ELISA. (d, e) Ctrl T, EGFRvIII CAR T in the presence of 0 and 145 ng/ml of mIL-10, or IL-10 EGFRvIII CAR T cells co-culture with 4T1-Luc-EGFRvIII cells at the E:T ratio of 0.5:1 for 48 h. (d) 4T1-Luc-EGFRvIII tumor cell killing percentage, and (e) viable CAR T cell counts were analyzed by flow cytometry. (f-h) BALB/c mice were injected i.v. with 4T1-Luc-EGFRvIII cells (5×10⁴) and received i.v. adoptive cell transfers of EGFRvIII CAR T cells (3×10⁶), IL-10 EGFRvIII CAR T cells (3×10⁶), or EGFRvIII CAR T cells (3×10⁶) followed by i.v. administration of mIL-10 (1 μg) on day 6, respectively. (f) Individual average radiance (p/s/cm2/sr) of groups of mice at different time points. (g) Shown are survival curves of each treatment group and the numbers of long-term-surviving mice among the total number of mice in the group. (h) Numbers of CAR T cells in blood on day 15, quantified by flow cytometry. Data represent the mean ±SEM.

FIG. 6—IL-10 Pmel TCR T therapy prolongs survival in mouse B16F10 melanoma model. (a) Transduction with IL-10 construct was conducted by retroviral vectors. The expression levels of IL-10 were analyzed by ELISA. (b-f) C57BL/6 mice were inoculated subcutaneously with B16F10 melanoma cells (3×10⁵) and received i.v. adoptive cell transfers of Pmel T cells (10×10⁶) or IL-10 Pmel T cells (10×10⁶) on day 6, respectively. (b-d) Individual tumor growth curves of PBS control group (b), Pmel T cell therapy (c) and IL-10 Pmel T cell therapy (d). (e, f) Shown are average tumor growth curves (e) and survival curves (f) of each treatment group. Data represent the mean ±SEM.

FIG. 7—in vitro characterization of IL-10 CD19 human CAR T. (a) Schematic representations of CD19-directed second generation CAR (CD19 CAR), and CD19-directed second generation CAR modified to express human IL-10 (IL-10 CD19 CAR). (b) The expression levels of CAR were analyzed by flow cytometry. Similar results were obtained from ten independent experiments. (c) The concentration of secreted IL-10 was detected by ELISA. (d) Ctrl T, CD19 CAR T, or IL-10 CD19 CAR T cells co-culture with Target tumor cells at the E:T ratio of 1:32, tumor cell killing percentage were analyzed by LDH assay. (e) Average tumor growth curves. NSG mice were inoculated (s.c.) with PANC1-CD19 human epithelioid carcinoma cells (2×10⁶) and received i.v. adoptive cell transfer of CD19 hCAR T cells (1×10⁶) or IL-10 CD19 hCAR T cells (1×10⁶) on day 8 (n=9 per group). Data represent the mean ±SEM.

FIG. 8—IL-10 expression sustains the mitochondrial fitness of CAR-T cells. C57BL/6 mice were inoculated with MC38-HER2 tumor cells (1×10⁶, s.c.), sublethally lymphodepleted by irradiation on day 5, and received i.v. adoptive transfer of IL-10 HER2 CAR-T cells (3×10⁶), or HER2 CAR-T cells (3×10⁶) in the presence or absence of i.v. administered IL-10 (1 μg) on day 6 (n=5 per group). On day 14, mice were killed and indicated tissues were processed. CAR-T cells were subjected to mitochondrial phenotype analysis by flow cytometry or sorted for electron microscope analysis. Mitochondrial mass and membrane potential of CAR-T cells were examined by staining with MitoTracker Green (MG) and MitoTracker Deep Red (MDR), respectively. a. Frequencies of HER2 CAR-T cells with dysfunctional mitochondria. b, The ratio of MDR/MG in tumor-infiltrating HER2 CAR-T cells from indicated treatment groups. c, Representative electron microscope images of sorted intratumoral HER2 CAR-T cells from indicated treatment groups. d-f, Quantification of mitochondrion number per cell (d), crista numbers per mitochondrion (e), and total crista length per mitochondrial area (f) in sorted intratumoral HER2 CAR-T cells as shown in (c). All data represent the mean ±s.e.m. and are analyzed by unpaired Student's t-test (d, e, f), or one-way ANOVA with Tukey's multiple-comparisons test (a, b).

FIG. 9—Mice Survivors post the treatment of IL-10 HER2 CAR-T or IL-10 TRP-1 CAR-T cells as described in FIG. 5 were rechallenged s.c. with MC38-HER2 (1×10⁶) and B16F10 (1×10⁵) cells, respectively, on day 90 post primary tumor inoculation. Naive WT mice (n=5 per group) were inoculated with the same number of tumor cells as controls. a, Experimental timeline. b,c, Shown are survival curves and numbers of long term survivors rejecting the second tumor challenge in MC38-HER2 (b) and B16F10 tumor models (c).

FIG. 10—Mice treated by IL-10 CAR-T cells induced stem cell-like memory. C57BL/6 mice were inoculated with MC38-HER2 cells (1×10⁶, s.c.), sublethally lymphodepleted by irradiation on day 5, and received i.v. adoptive transfer of IL-10 HER2 CAR-T cells (3×10⁶), or HER2 CAR-T cells (3×10⁶) in the presence or absence of i.v. administered IL-10 (1 μg) on day 6 (n=5 per group). On day 18, mice were killed for phenotype analyses of CAR-T cells in spleen and peripheral blood by flow cytometry. a,b, Average frequencies of CD62L⁺CD44⁻ among total CAR-T cells in spleen (a) and Sca-1⁺CD122⁺ among CD62L⁺CD44⁻ CAR-T cells (b). c, Representative flow cytometry plots and average MFI of Sca-1 expression in CAR-T cells in spleen. d, Representative flow cytometry plots showing phenotypes of CAR-T cells in spleen and blood (e). Frequencies of IL-7Rα⁺KLRG-1⁻ among total CAR-T cells in spleen (d) and blood (e). All data represent the mean ±s.e.m. and are analyzed by one-way ANOVA with Tukey's multiple-comparisons test (a-c).

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of “include/including”, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc. For example, at least one means one or more constructs and refers to one construct, two constructs, three constructs, etc . . .

As used herein the terms “subject” /“subject in need thereof”, or “patient” /“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human that might be at risk of suffering from a cancer.

According to the present invention, the cancer is a solid or a liquid cancer. In one aspect, the cancer is a solid cancer. Preferably, the solid cancer is selected from the non-limiting group comprising lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer, brain cancer and skin cancer, in particular melanoma, or a combination of one or more thereof.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single-or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity, i.e., an analogue of A will base-pair with T.

The term “vector”, as used herein, refers to a viral vector or to a nucleic acid (DNA or RNA) molecule such as a plasmid or other vehicle, which contains one or more heterologous nucleic acid sequence(s) of the invention and, preferably, is designed for transfer between different host cells and/or for amplification purposes.

The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

As used herein, Interleukine-10 (IL-10), refers to a member of the IL-10 family cytokines. IL-10 is generally considered immunosuppressive as it reduces tissue damage caused by uncontrolled inflammatory responses. “IL-10, a fragment or a variant thereof” include sequences comprising the sequence of, preferably, native human IL-10 as well as fragment and variants thereof such as described in Mumm et al., 2011, Cancer Cell, 20, 781-796; Guo et al., 2012, Protein Expr. Purif., 83, 152-156 (2012); Zheng et al., 1997, J. Immunol., 158, 4507-13; Qiao et al., 2019, Cancer Cell 35, 901-915; Guo et al., 2021, Nat Immunol 22, 746-756 the contents of which are hereby incorporated by reference in their entirety. In one aspect, the IL-10 sequence is a human IL-10 amino acid sequence as set forth in SEQ ID No: 1.

The term “variant”, when it refers to IL-10, means one or more biologically active derivatives of an IL-10, preferably of a human IL-10 sequence of the invention. In general, the term “variant” refers to molecules having a native sequence with one or more additions, substitutions (generally conservative in nature) and/or deletions, relative to the native molecule, so long as the modifications do not destroy its biological activity and which are “substantially homologous” to the reference molecule (Gorby et al., Sci. Signal. 13, eabc0653, 2020; Saxton et al., Science 371, eabc8433, 2021). In general, the sequences of such variants will have a high degree of sequence homology or identity to the reference sequence, e.g., sequence homology or identity of more than 25%, generally more than 50% to 70%, even more particularly 80%, or 85% or more, such as at least 90%, or 95% or more, when the two sequences are aligned. Spencer, Juliet V et al. reported that splicing forms of IL-10 retain biological activities or properties, despite having only 27% sequence identity to hIL-10 (Spencer, Juliet V et al. “Stimulation of B lymphocytes by cmvIL-10 but not LAcmvIL-10.” Virology vol. 374,1 (2008): 164-9. doi: 10.1016/j.virol.2007.11.031, the contents of which are hereby incorporated by reference in their entirety).

As used herein, a “fragment” of an IL-10, preferably of a human IL-10, of the invention refers to a sequence containing less nucleotides in length than the respective polypeptide sequence or nucleic acid sequence. Preferably, this sequence or fragment contains less than 90%, preferably less than 60%, in particular less than 30% nucleotides in length than the respective polypeptide sequence or nucleic acid sequence.

While focusing on developing novel and efficient approaches for treating tumors with CAR engineered T cells, the Inventors surprisingly showed that metabolic engineered IL-10 expressing CAR T redirects CAR T cell fate away from exhaustion and towards a memory-like state, leading to eradication of established solid tumors and durable cures in the majority of treated mice. These promising results evidenced the great potential of IL-10 expressing CAR T or any other engineered immune cells to enhance the efficacy of adoptive cell therapy in the clinic.

The present invention provides, in one aspect, an immune cell, or a population of immune cells, expressing an interleukin-10, a fragment or a variant thereof. In one aspect the immune cell is an isolated immune cell.

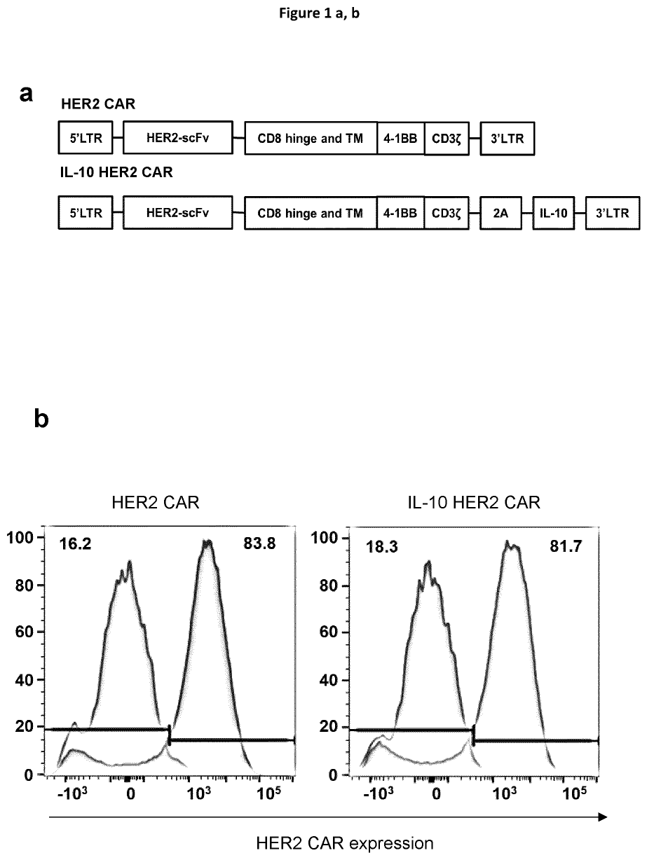

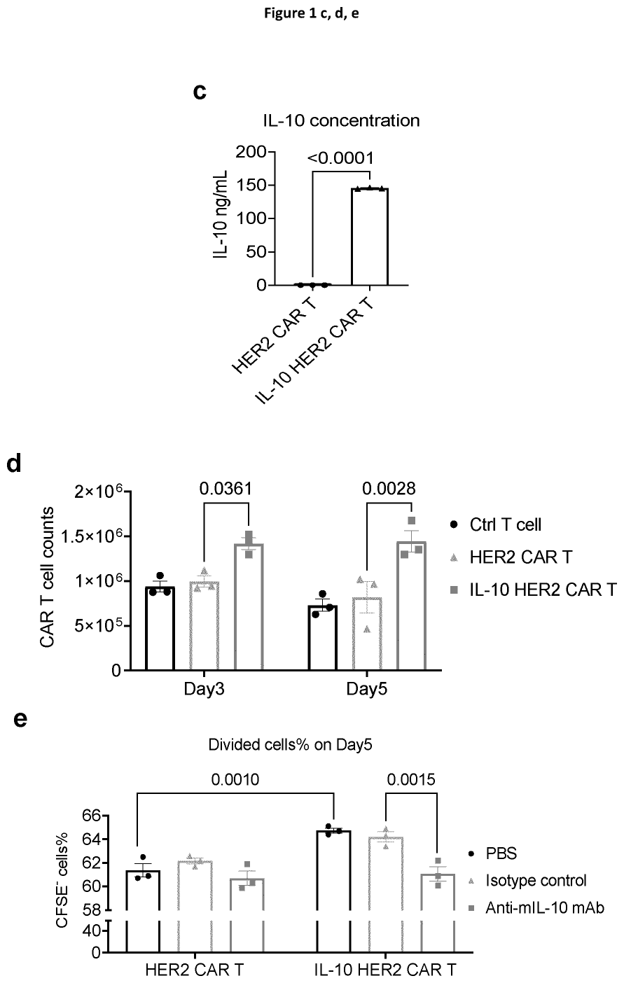

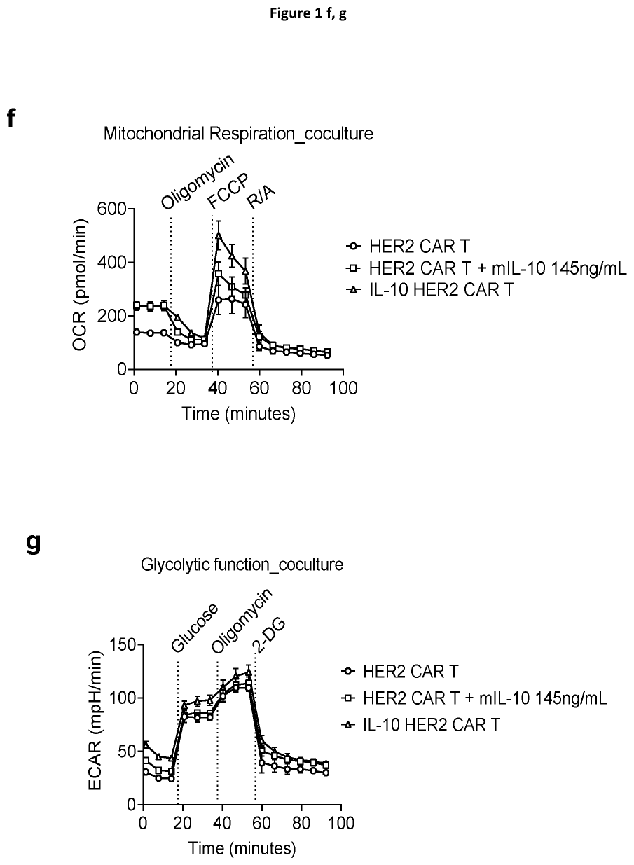

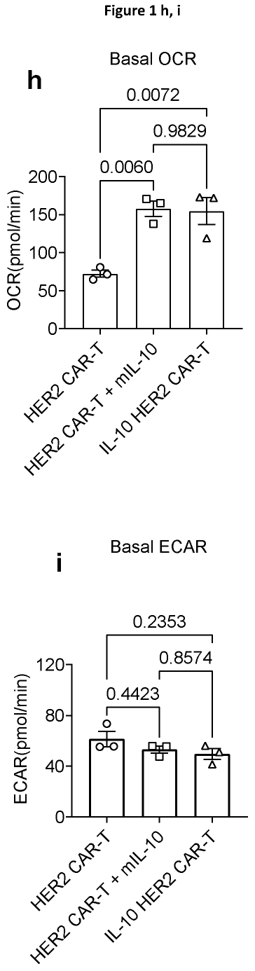

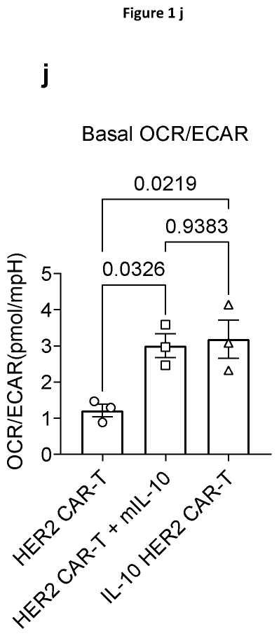

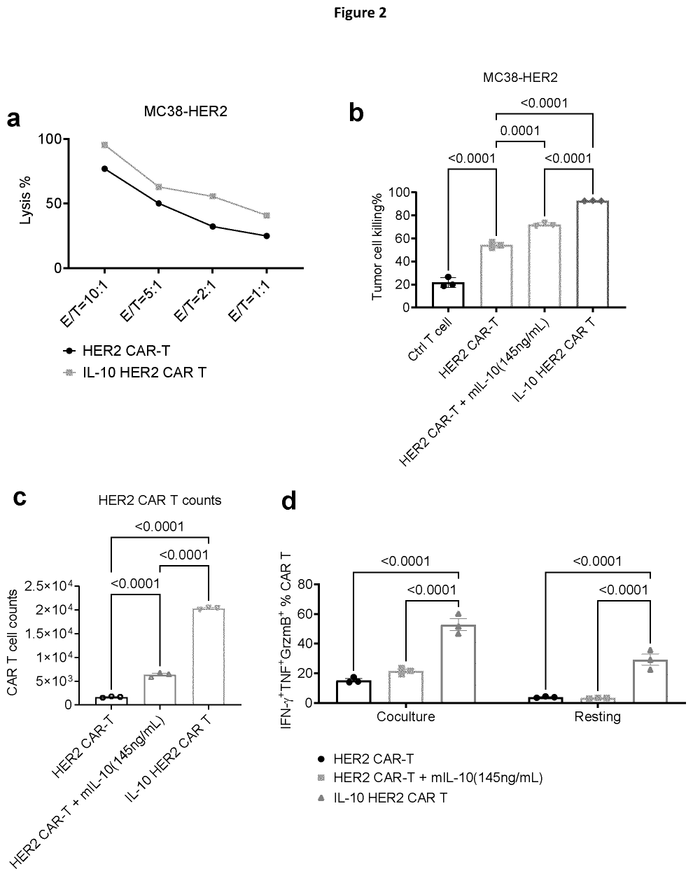

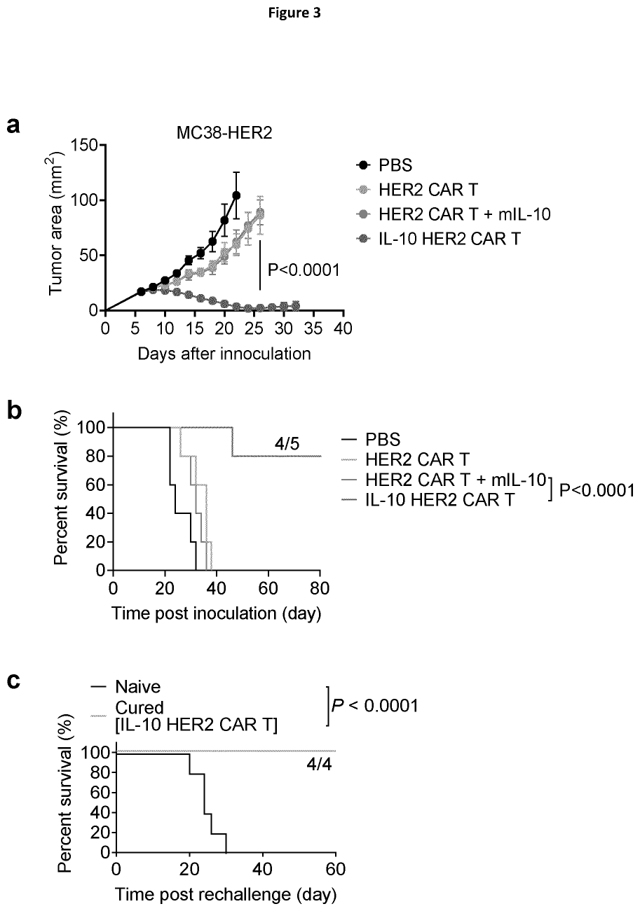

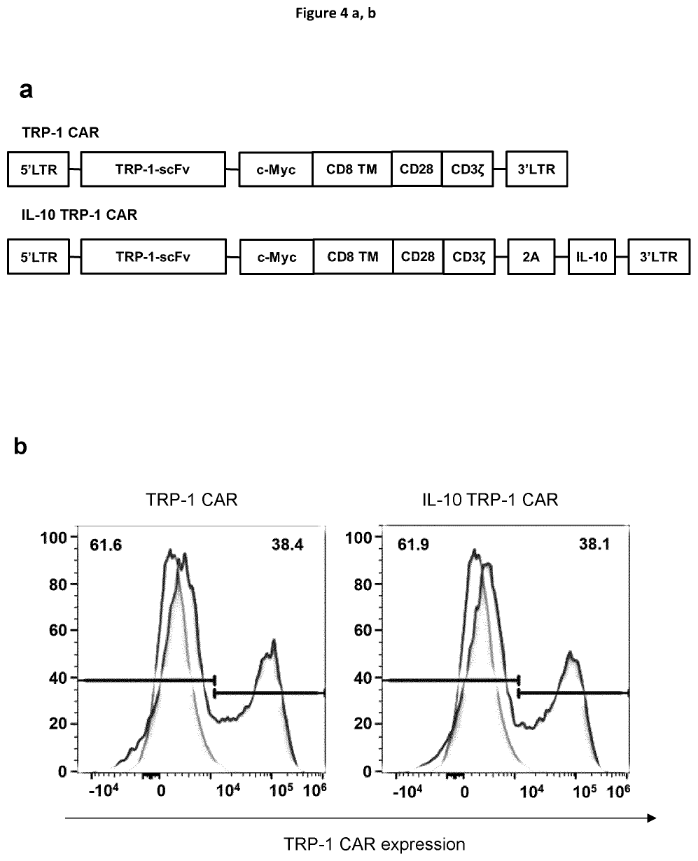

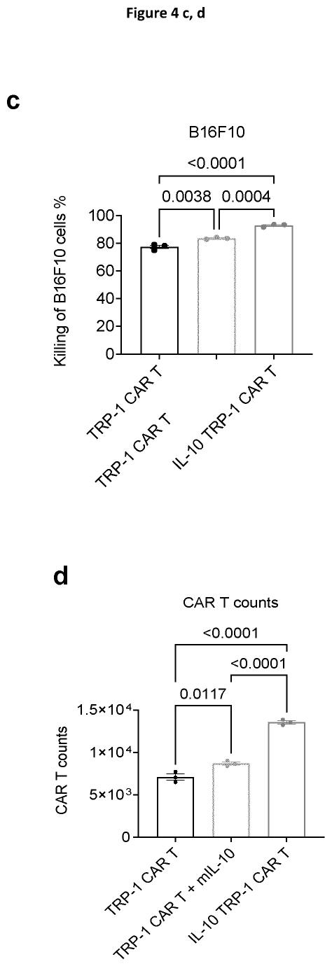

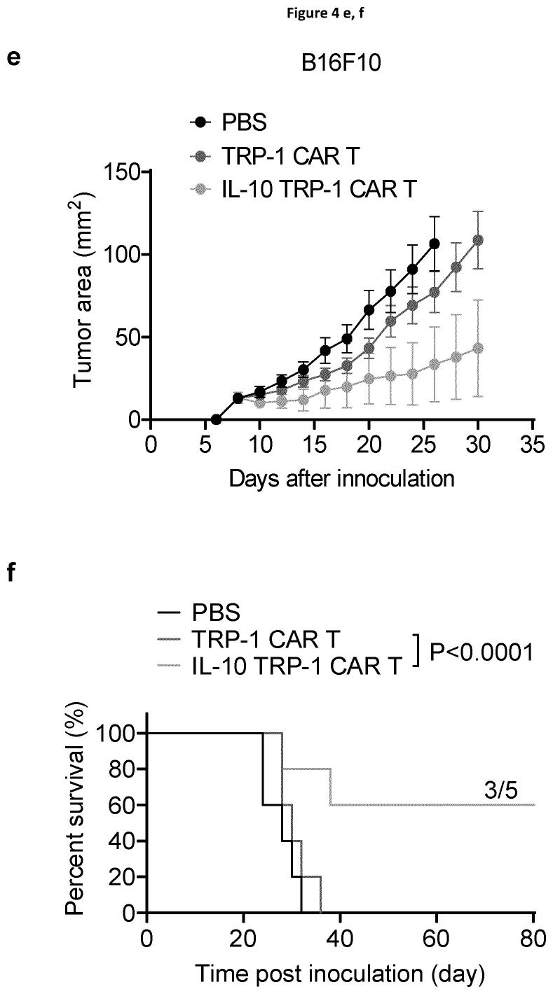

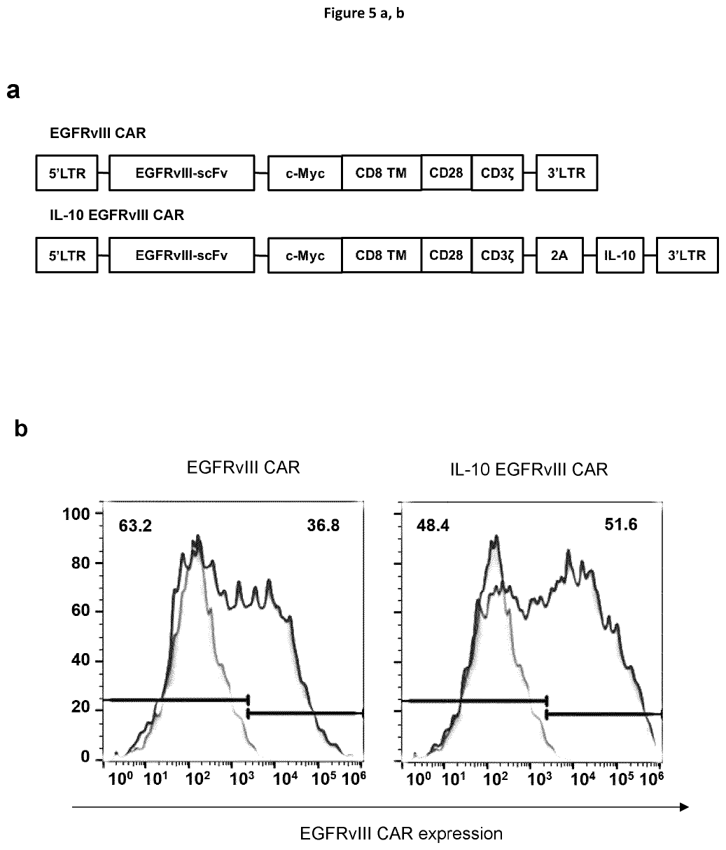

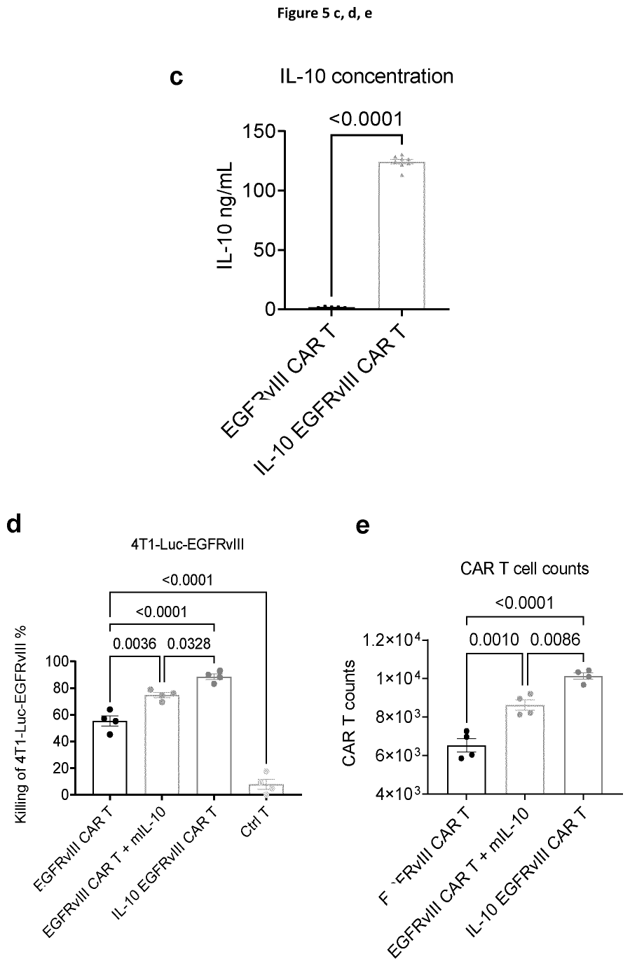

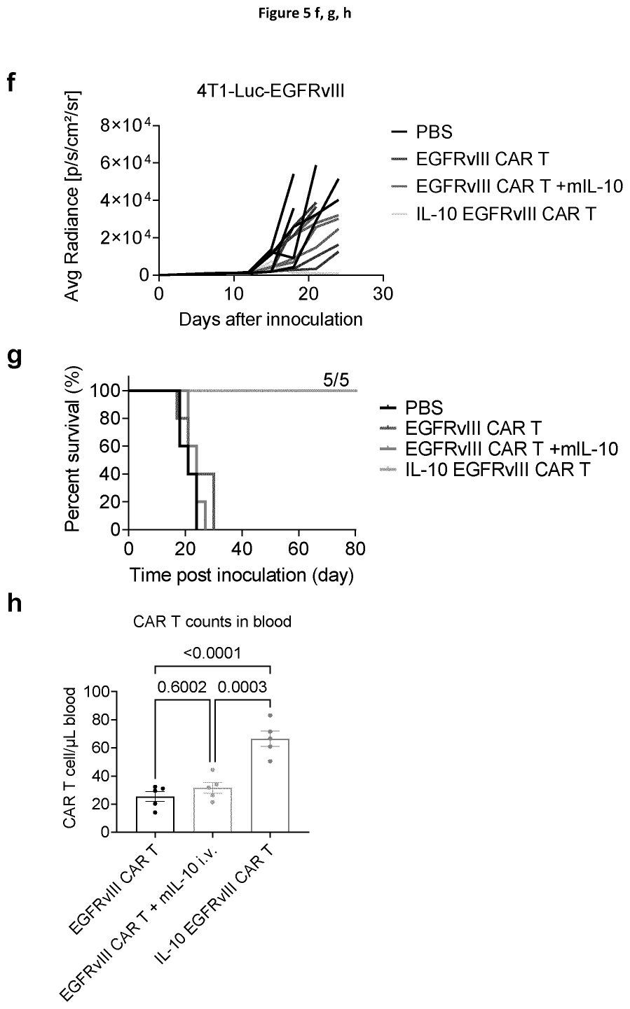

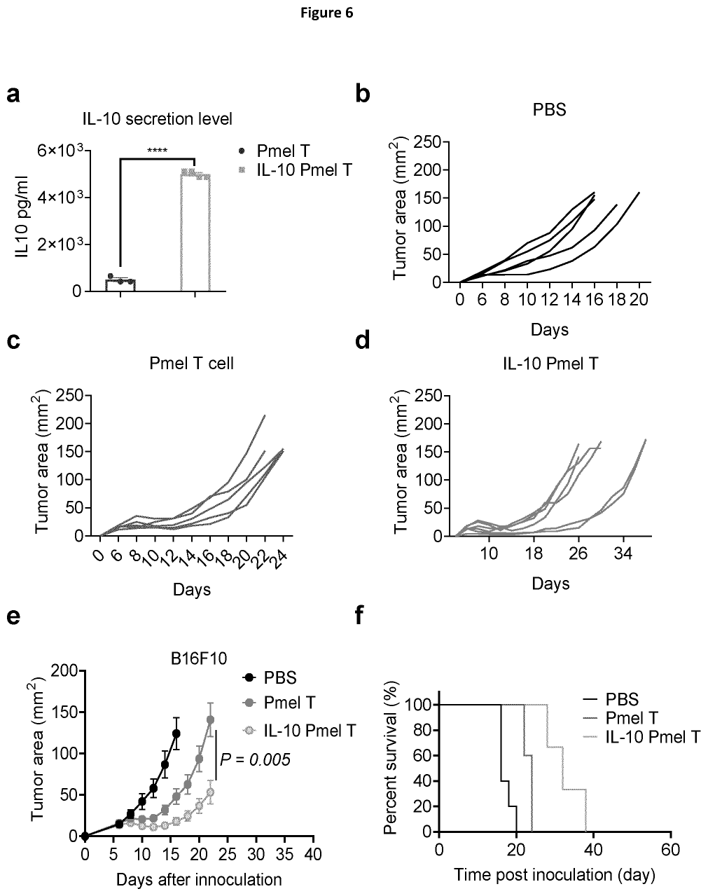

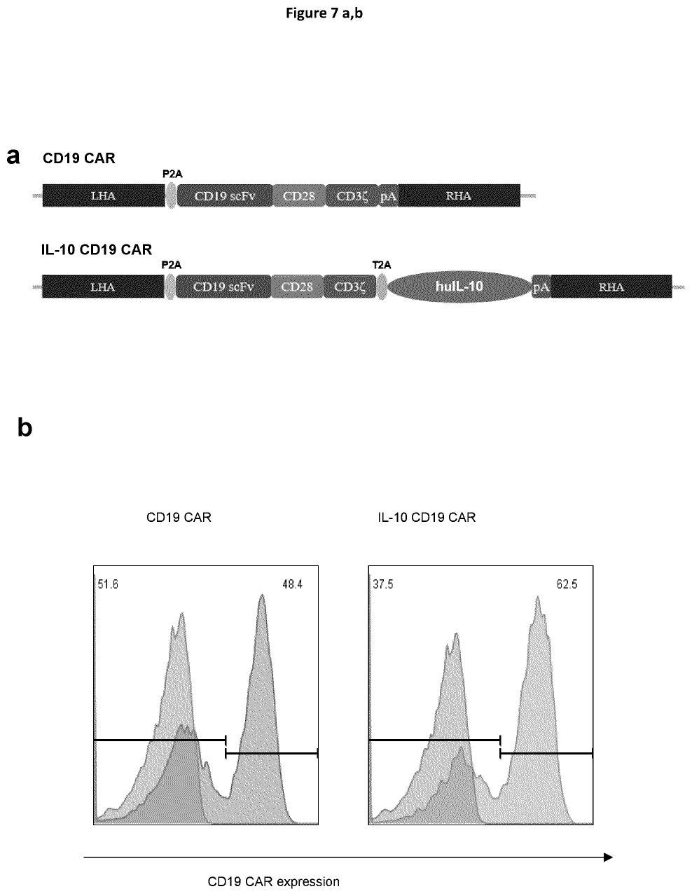

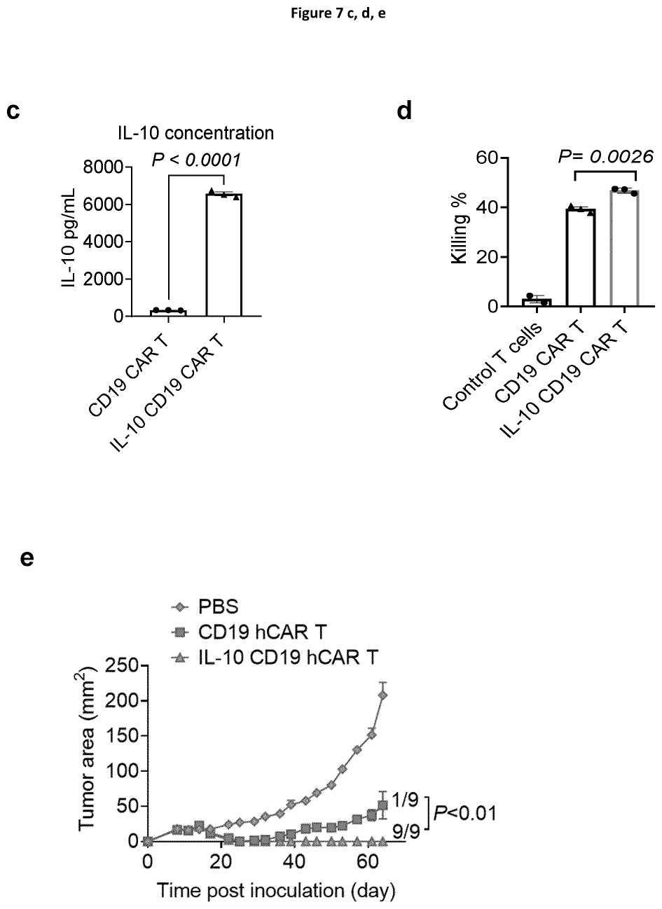

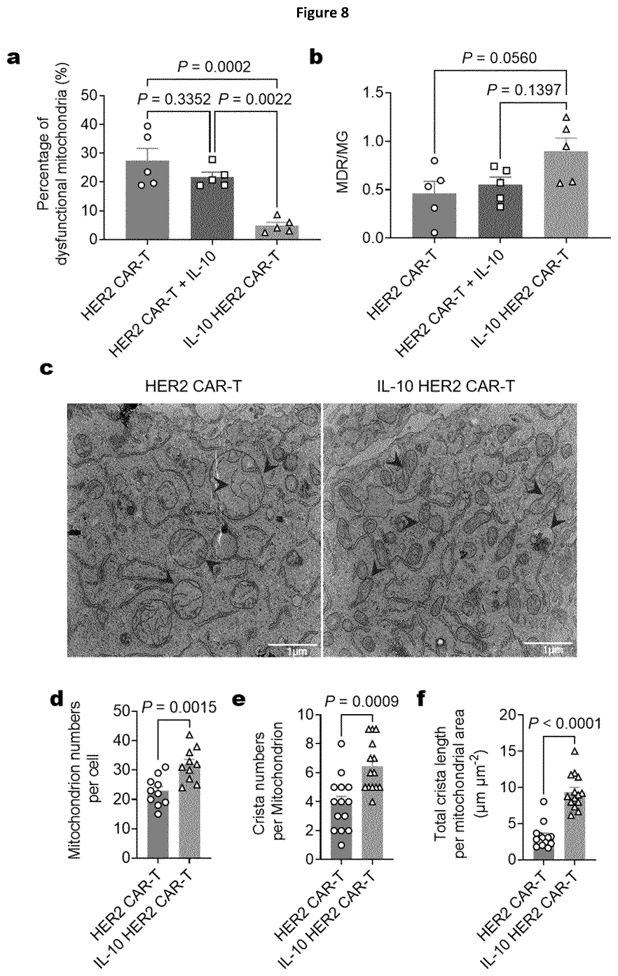

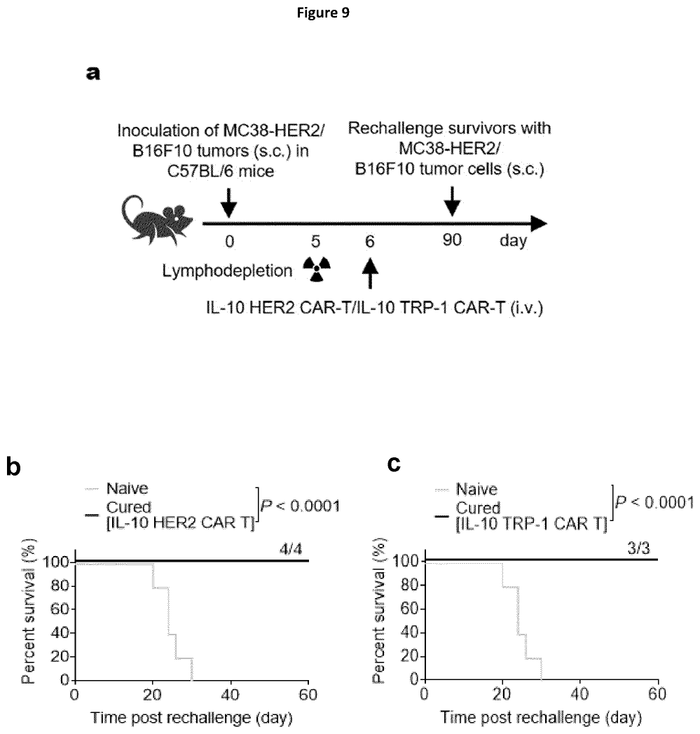

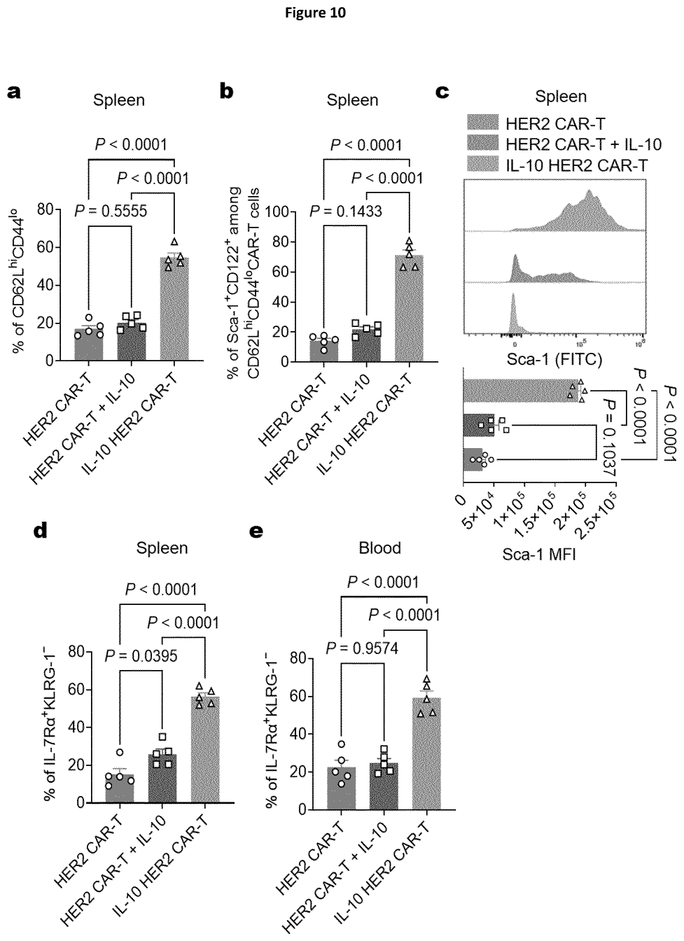

As used herein, the term “immune cell” includes any type of immune cells categorized as lymphocytes, neutrophils, and monocytes/macrophages, whether recombinant (engineered) or not. In a preferred aspect, the immune cell is selected among the non-limiting group comprising T cell, chimeric antigen receptor (CAR)-T cell, T cell receptor (TCR)-transgenic T cell, tumor infiltrating lymphocyte (TIL), NK cell, NK-T cell, CAR-NK cell, CAR-NKT cell, TCR-transgenic NK cell, TCR-transgenic NK-T cell, dendritic cell, macrophage, CAR-macrophage or any synthetic tumor specific immune cells. The immune cells population of immune cells can be
autologous, i.e. using patient's own immune cells,
allogeneic, i.e. obtained from donor blood, umbilical cord blood or pluripotent stem cells (such as iPSCs that can be genetically engineering), or
heterologous.
   In the two last cases, strategies to reduce allo-rejection are also to be considered and are knoon to the skilled artisan.
   Preferably, the immune cell comprises one or more recombinant constructs, wherein at least one recombinant construct encodes an interleukin-10, a fragment or a variant thereof.
   In one aspect, the construct encoding an interleukin-10, a fragment or a variant thereof comprises or encodes an amino acid sequence of SEQ ID No. 1, or a fragment or variant thereof.
   In one aspect, the recombinant construct encoding an interleukin-10, a fragment or a variant thereof is linked to the second recombinant construct encoding a CAR, a TCR, or any other synthetic tumor targeting motif.
   Preferably also, the second recombinant construct encodes a chimeric antigen receptor (CAR), a T cell receptor (TCR) or any other synthetic tumor targeting motif. Non-limiting examples of synthetic tumor-targeting motifs comprise, e.g. tumor-targeting peptides listed in Tables 2 and 3 of Liu R, Li X, Xiao W, Lam KS (Tumor-targeting peptides from combinatorial libraries [published correction in Adv Drug Deliv Rev. 2018 Mar. 9], which are hereby incorporated by reference in their entirety.
   One skilled in the art knows that tumor-targeting motifs, such as e.g. tumor-targeting peptides, can be detected from, e.g. phage-display libraries via screening approaches selected from in vitro, in vivo or ex vivo selections approaches (see for example FIG. 1 of Liu R, Li X, Xiao W, Lam KS). Cancer-associated proteins, specific cancer cell lines, patient tissues, and tumor xenograft mouse models can be used as screening probes.
   Preferably, said linking is via a sequence encoding a self-cleaving peptide (e.g. peptide 2A, see e.g. Takahashi, K. & Yamanaka, S. Induction of pluripotent stem cells from mouse embryonic and adult fibroblast cultures by defined factors. Cell 126, 663-676, which is hereby incorporated by reference in its entirety or an internal ribosome entry site (IRES) sequence). For example, once the self-cleaving peptide is cleaved, IL-10, fragment or variant thereof, is secreted or membrane bond by, or on, the immune cell, preferably in the tumor microenvironment.

In one aspect, the recombinant construct comprises a nucleic acid encoding the CAR that encodes an extracellular antigen recognition domain of the single-chain Fragment variant (scFv), a polypeptide of a transmembrane region, an intracellular T cell activation domain and/or an intracellular region.

The extracellular antigen recognition domain of the single-chain Fragment variant (scFv) is, preferably, derived from an antibody or a ligand or a receptor. In some instances, the extracellular domain comprises a hinge portion. A variety of hinges can be employed in accordance with the invention, such as e.g. CD8 hinge.
Usually, the extracellular antigen recognition domain of the single-chain fragment variant (scFv) derived from an antibody recognizes an antigen selected from the non-limiting group comprising c-MET, TRP-1, CD19, CD20, BCMA, CD133, CD171, CD70, CEA, EGFR, EGFR-vIII, EpCAM, EphA2, FAP, GD2, GPC3, HER2, HER3, IL-13Ra2, Mesothelin, MUC1, Claudin 18.2, PSCA, PSMA, ROR1, and VEGFR2 or a combination of one or more thereof. In a preferred aspect, the extracellular antigen recognition domain in the CAR of the invention is a CD8 or CD28 transmembrane domain scFv, e.g. linked to a hinge, that recognizes HER2 (SEQ ID NO: 2), TRP-1 (SEQ ID NO: 3), EGFR-vIII. (SEQ ID NO: 4) or CD19 (SEQ ID NO: 5).
The transmembrane region and hinge is usually fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (comprise, or correspond to) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CDl-la/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLAI, CD49a, ITGA4, IA4, CD49D, ITGA6, VLA-6, CD49f, ITGAD, CDI ld, ITGAE, CD103, ITGAL, CDI la, LFA-1, ITGAM, CDI 1b, ITGAX, CDI Ic, ITGBI, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.
Optionally, short linkers may form linkages between any or some of the extracellular, transmembrane, and intracellular domains of the CAR.
In a preferred aspect, the transmembrane domain and hinge in the CAR of the invention is a CD8 transmembrane domain and hinge. In one aspect, the CD8 transmembrane domain and hinge comprises the transmembrane portion and hinge of the amino acid sequence of SEQ ID NO: 6, a fragment or a variant thereof.
The intracellular T cell activation domain is capable of activating the T cell upon binding of the antigen binding molecule to its target. It will be appreciated that the intracellular domain typically further comprises one or more costimulatory molecules as described herein.
In further aspects, the T cell activation domain comprises CD3, preferably CD3 zeta, more preferably CD3 zeta (CD35) of the amino acid sequence of SEQ ID NO: 7, a fragment or a variant thereof.
A “costimulatory molecule” as used herein refers to a molecule that provides a signal which mediates a T cell response, including, but not limited to, proliferation, activation, differentiation, and the like. Costimulatory molecules can provide a signal in addition to the primary signal provided by an activating molecule as described herein.
The intracellular (cytoplasmic) region of the engineered T cells of the invention can provide activation of at least one of the normal effector functions of the immune cell. Effector function of a T cell, for example, may refer to cytolytic activity or helper activity.
It will be appreciated that suitable intracellular region include (i.e., comprise), but are not limited to signaling domains derived from (or corresponding to) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CDl-la/CD18), CD3gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDI ld, ITGAE, CD103, ITGAL, CDl la, LFA-1, ITGAM, CDI 1b, ITGAX, CDl lc, ITGBI, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.
An example of a combination comprises a 4-1BB and a CD28 intracellular region.
In a preferred aspect, the intracellular domain of the CAR comprises a 4-1BB intracellular region.
Exemplary CAR constructs in accordance with the invention are as set forth in FIGS. 1a, 4a, and 7a.
In case the second recombinant construct encodes a transgenic TCR, said TCR preferably recognizes an antigen selected from the non-limiting group comprising gp100, NY-ESO-1, MAGE-A3 and TRP-1, or a combination of one or more thereof.
In one aspect, the construct encoding an interleukin-10, a fragment or a variant thereof is comprised within a sequence encoding a Fc, human serum albumin (HSA), or antibody fusion protein.

In an aspect of the invention, the immune cell, or population of immune cells, described herein is for use in the prevention and/or treatment of cancer. The cancer can be either a solid or a liquid cancer.

Preferably, the cancer is a solid cancer selected from the non-limiting group comprising lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer, brain cancer, lymphoma and skin cancer, in particular melanoma, or a combination of one or more thereof. More preferably the solid cancer is selected from the group comprising lymphoma, breast cancer, gastric cancer and melanoma.

The present invention further provides a nucleic acid sequence encoding one or more recombinant constructs described herein, including the SEQ IDs disclosed herein.

The present invention further provides a plasmid or a vector comprising a nucleic acid sequence encoding one or more recombinant constructs described herein, including the SEQ IDs disclosed herein.

Any vector known in the art can be suitable for the present invention. In some aspects, the vector is a viral vector. In some aspects, the vector is a retroviral vector (such as pMSGV), a DNA vector, a murine leukemia virus vector, an SFG vector, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector (such as pGAR), or any combination thereof.

The present invention also contemplates compositions as well as pharmaceutical compositions.

In an aspect of the invention, the pharmaceutical composition of the invention comprises a therapeutically effective amount of i) an immune cell, or population of immune cells, described herein, ii) a nucleic acid described herein, and/or iii) a plasmid or a vector described herein, and at least one pharmaceutically acceptable carrier and/or diluent.

The term “therapeutically effective amount” as used herein means an amount of an immune cell, nucleic acid, plasmid or vector, high enough to significantly positively modify the symptoms and/or condition to be treated, but low enough to avoid serious side effects (at a reasonable risk/benefit ratio), within the scope of sound medical judgment.
The therapeutically effective amount of an immune cell, nucleic acid, plasmid or vector as described herein is selected in accordance with a variety of factors including type, species, age, weight, sex and medical condition of the patient or subject; the severity of the condition or disease (e.g. cancer) to be treated; the route of administration; the renal and hepatic function of the patient or subject. A physician of ordinary skill in the art can readily determine and prescribe the effective amount of the immune cell, nucleic acid, plasmid of vector required to prevent, counter or arrest the progress of the disease, such as e.g. cancer.
“Pharmaceutically acceptable carrier or diluent” means a carrier or diluent that is useful in preparing a pharmaceutical composition that is generally safe, non-toxic, and desirable, and includes carriers or diluents that are acceptable for human pharmaceutical use.
The immune cells or population of immune cells of the present invention may be administered either alone, or as a pharmaceutical composition. Pharmaceutical compositions of the present invention may comprise the immune cells or population of cells, such as T cells, as described herein, in combination with one or more pharmaceutically or physiologically acceptable carriers or diluents. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.
The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono-or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.
The pharmaceutical composition of the invention can further comprise at least one additional therapeutic agent or therapy. A variety of other additional therapeutic agents may be used in conjunction with the compositions described herein.
In one aspect, said at least one additional therapeutic agent or therapy is an anticancer agent or anticancer therapy, useful to treat a cancer, preferably a solid cancer. Preferably, the one or more anti-cancer therapy will be selected from the group comprising radiotherapy, chemotherapy, immune checkpoint inhibitor, immunotherapy and hormone therapy, or a combination of one of more thereof.
Preferably, the immune checkpoint inhibitor is selected from the group comprising a PD-1 inhibitor, a PD-L1 inhibitor, and a CTLA-4 inhibitor, or a combination of one of more thereof.
For example, potentially useful additional therapeutic agents include PD-1 inhibitors such as nivolumab (Opdivo®), pembrolizumab (Keytruda®), pembrolizumab, pidilizumab, and atezolizumab.
For example, potentially useful additional therapeutic agents include PD-L1 inhibitors such as atezolizumab, avelumab, AMP-224, MEDI-0680, RG-7446, GX-P2, durvalumab, KY-1003, KD-033, MSB-0010718C, TSR-042, ALN-PDL, STI-A1014, CX-072, and BMS-936559.
Non-limiting examples of CTLA-4 inhibitors include ipilimumab (Yervoy) (also known as BMS-734016, MDX-010, MDX-101) and tremelimumab (formerly ticilimumab, CP-675,206).
A chemotherapy of the present invention can concern agents that damage DNA and/or prevent cells from multiplying, such as genotoxins.
Genotoxins can be selected from the group comprising alkylating agents, antimetabolites, DNA cutters, DNA binders, topoisomerase poisons and spindle poisons. Examples of alkylating agents are lomustine, carmustine, streptozocin, mechlorethamine, melphalan, uracil nitrogen mustard, chlorambucil, cyclosphamide, iphosphamide, cisplatin, carboplatin, mitomycin, thiotepa, dacarbazin, procarbazine, hexamefhyl melamine, triethylene melamine, busulfan, pipobroman, mitotane and other platine derivatives.
An example of DNA cutters is bleomycin.
Topoisomerases poisons can be selected from the group comprising topotecan, irinotecan, camptothecin sodium salt, daorubicin, doxorubicin, idarubicin, mitoxantrone teniposide, adriamycin and etoposide.
Examples of DNA binders are dactinomycin and mithramycin whereas spindle poisons can be selected among the group comprising vinblastin, vincristin, navelbin, paclitaxel and docetaxel.
A chemotherapy of the present invention can concern antimetabolites selected among the following coumpounds: methotrexate, trimetrexate, pentostatin, cytarabin, ara-CMP, fludarabine phosphate, hydroxyurea, fluorouracyl, fioxuridine, chlorodeoxyadenosine, gemcitabine, thioguanine and 6-mercaptopurine.
Radiotherapy refers to the use of high-energy radiation to shrink tumors and kill cancer cells. Examples of radiation therapy include, without limitation, external radiation therapy and internal radiation therapy (also called brachytherapy).
External radiation therapy is most common and typically involves directing a beam of direct or indirect ionizing radiation to a tumor or cancer site. While the beams of radiation, the photons, the Cobalt or the particule therapy are focused to the tumor or cancer site, it is nearly impossible to avoid exposure of normal, healthy tissue. Energy source for external radiation therapy is selected from the group comprising direct or indirect ionizing radiation (for example: x-rays, gamma rays and particle beams or combination thereof).
Internal radiation therapy involves implanting a radiation-emitting source, such as beads, wires, pellets, capsules, etc., inside the body, at, or near to the tumor site. Energy source for internal radiation therapy is selected from the group of radioactive isotopes comprising: iodine (iodine125 or iodine131), strontium89, radioisotopes of phosphorous, palladium, cesium, indium, phosphate, or cobalt, and combination thereof. Such implants can be removed following treatment, or left in the body inactive. Types of internal radiation therapy include, but are not limited to, interstitial, and intracavity brachytherapy (high dose rate, low dose rate, pulsed dose rate).
A currently less common form of internal radiation therapy involves biological carriers of radioisotopes, such as with radio-immunotherapy wherein tumor-specific antibodies bound to radioactive material are administered to a patient or subject. The antibodies bind tumor antigens, thereby effectively administering a dose of radiation to the relevant tissue.
Methods of administering radiation therapy are well known to those of skill in the art.
A variety of other additional therapeutic agents may be used in conjunction with the compositions described herein.
Additional therapeutic agents suitable for use in combination with the invention include, but are not limited to, ibrutinib (Imbruvica®), ofatumumab (Arzerra®), rituximab (Rituxanx®), bevacizumab (Avastin®), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).
In additional aspects, the additional therapeutic agent can be an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRAR®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

The present invention further contemplates methods of treatment and/or prevention of a cancer.

The term “treatment” or “treating” means any administration of a composition, pharmaceutical composition, therapeutic agent, compound, etc . . . of the disclosure to a subject for the purpose of:
(i) inhibiting the disease, that is, arresting the development of clinical symptoms; and/or
(ii) relieving the disease, that is, causing the regression of clinical symptoms.
   As used herein, the term “prevention” or “preventing” means any administration of a composition, pharmaceutical composition, therapeutic agent, compound, etc . . . of the disclosure to a subject for the purpose of preventing the disease, that is, causing the clinical symptoms of the disease not to develop.
   In the context of the present invention, the disease is a cancer, preferably a solid tumor as disclosed herein.
   In one aspect, the method of treatment and/or prevention of a cancer in a patient or subject comprises (i) removing and isolating immune cells, preferably native T cells, from said patient or subject, (ii) genetically engineering said T cells with one recombinant construct encoding an interleukin-10, a fragment or a variant thereof and with a second recombinant construct encoding a chimeric antigen receptor (CAR), a T cell receptor (TCR) or any other synthetic tumor targeting motif or antigen, (iii) expanding ex vivo into a larger population of engineered T cells, and (iv) reintroducing said engineered T cells, into the patient or subject. After the engineered T cells are reintroduced into the patient or subject, they mediate an immune response against cells expressing the tumor targeting motif or antigen described herein. This immune response includes secretion of IL-10, a fragment or a variant thereof, and other cytokines by T cells, the clonal expansion of T cells recognizing the tumor targeting motif or antigen, and T cell-mediated specific killing of target-positive cells.
   In one aspect the method of treatment and/or prevention of a cancer comprises (i) removing and isolating immune cells, preferably native T cells, from a patient or subject, or providing immune cells, preferably native T cells, (ii) genetically engineering said T cells with at least one recombinant construct encoding an interleukin-10, a fragment or a variant thereof and with a second recombinant construct encoding a chimeric antigen receptor (CAR), a T cell receptor (TCR) or any other synthetic tumor targeting motif or antigen, (iii) expanding ex vivo into a larger population of engineered T cells, and (iv) reintroducing into the patient or subject.
   In one aspect the method of treatment and/or prevention of a cancer in a subject comprises administering a pharmaceutical composition of the invention to a subject in need thereof.
   In one aspect, the methods of treatment and/or prevention described above, can further comprise administrating at least one additional therapeutic agent or therapy, preferably an anticancer agent or anticancer therapy, more preferably a therapeutically effective amount or dose of an anticancer agent or anticancer therapy. Said one or more anti-cancer agent or therapy will be selected among the non-limiting group comprising radiotherapy, chemotherapy, immune checkpoint inhibitor, immunotherapy and hormone therapy, or a combination of one of more thereof, as described supra.

A variety of known techniques can be utilized in making the polynucleotides, polypeptides, vectors, antigen binding molecules, immune cells, compositions, and the like according to the invention.

Prior to the in vitro manipulation or genetic modification of the immune cells described herein, the cells may be obtained and isolated from a subject. In some aspects, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain aspects, T cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used. After washing, the cells may be resuspended in a variety of biocompatible buffers, or other saline solution with or without buffer. In certain aspects, the undesired components of the apheresis sample may be removed.
In certain aspects, T cells are isolated from PBMCs by lysing the red blood cells and depleting the monocytes, for example, using centrifugation through a PERCOLL™ gradient. A specific subpopulation of T cells, such as CD28⁺, CD4⁺, CD8⁺, CD45RA⁺, and CD45RO⁺ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4 cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.
PBMCs may be used directly for genetic modification with the immune cells (such as CARs or TCRs) using methods as described herein. In certain aspects, after isolating the PBMCs, T lymphocytes can be further isolated and both cytotoxic and helper T lymphocytes can be sorted into naive, memory, and effector T cell subpopulations either before or after genetic modification and/or expansion.
In some aspects, CD8⁺ cells are further sorted into naive, central memory, and effector cells by identifying cell surface antigens that are associated with each of these types of CD8⁺ cells.
The immune cells described herein can be genetically modified following isolation using known methods, or the immune cells can be activated and expanded (e.g. TIL cells) or differentiated in the case of progenitors in vitro prior to being genetically modified. In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory molecule and a costimulatory molecule, such as anti-CD3 and anti-CD28 antibodies, generally attached to a bead or other surface, in a culture medium with appropriate cytokines, such as IL-2. In other aspects, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.
Certain methods for making the constructs and engineered immune cells of the invention are described in, e.g. PCT application PCT/US2015/14520, the contents of which are hereby incorporated by reference in their entirety.
It will be appreciated that PBMCs can further include other cytotoxic lymphocytes such as NK cells or NKT cells. An expression vector carrying a recombinant construct of the invention as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one aspect, the in vitro transduction, culture and/or expansion of T cells are performed in the absence of non-human animal derived products such as fetal calf serum and fetal bovine serum.
For cloning of polynucleotides of the invention, the vector may be introduced into a host cell (autologous, allogeneic or heterologous) to allow replication of the vector itself and thereby amplify the copies of the polynucleotide contained therein. The cloning vectors of the invention may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.
The term “autologous” refers to any material derived from the same individual to which it is later to be re-introduced.
The term “allogeneic” refers to any material derived from one individual which is then introduced to another individual of the same species, e.g., allogeneic T cell transplantation.

In certain aspects, the present disclosure provides isolated host cells containing the vector provided herein. The host cells containing the vector may be useful in expression or cloning of the polynucleotide contained in the vector. Suitable host cells can include, without limitation, oncolytic virus, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art.
Also contemplated is a method of enhancing antitumor activity in a subject comprising (i) removing and isolating immune cells, preferably native T cells, from said subject, or providing immune cells, preferably native T cells, (ii) genetically engineering said T cells with at least one recombinant construct encoding an interleukin-10, a fragment or a variant thereof and with a second recombinant construct encoding a chimeric antigen receptor (CAR), a T cell receptor (TCR) or any other synthetic tumor targeting motif or antigen, (iii) expanding ex vivo into a larger population of engineered T cells, and (iv) reintroducing into the patient or subject.

Also contemplated is a kit for performing one or more methods according to the invention.

Further contemplated is a kit comprising a composition or a pharmaceutical composition of the invention in one or more containers. Compositions can be in liquid form or can be frozen. Suitable containers for the compositions include, for example, bottles, vials, syringes, and test tubes. Containers can be formed from a variety of materials, including glass or plastic. The kit may further contain instructions that may include information or directions, drug quantity, composition, and so forth for the prescription.

Those skilled in the art will appreciate that the invention described herein is not limited to the use of IL-10 expressing immune cell (such as CAR T or TCR T) transfer therapy for treating cancer. As IL-10 expressing CAR T cells could be considered as one example strategy for tumor targeted delivery of IL-10 which enhanced anti-tumor immunity, one can expect that tumor targeted delivery of IL-10 by other strategies, such as stem cells (Liu, L., et al., Mechanoresponsive stem cells to target cancer metastases through biophysical cues. Sci. Transl. Med. 9, eaan2966 (2017), blood platelets (Wang, C.et al., In situ activation of platelets with checkpoint inhibitors for post-surgical cancer immunotherapy. Nat. Biomed. Eng. 1, (2017)), oncolytic virus (Rivadeneira, D. B.et al., Oncolytic Viruses Engineered to Enforce Leptin Expression Reprogram Tumor-Infiltrating T Cell Metabolism and Promote Tumor Clearance. Immunity 51, 548-560. e4 (2019)), mRNA (cancer vaccine, see e.g. Sahin, U., et al., Personalized RNA mutanome vaccines mobilize poly-specific therapeutic immunity against cancer. Nature 547, 222-226 (2017)) or nanotechnology (Tang, L.et al., Enhancing T cell therapy through TCR-signaling-responsive nanoparticle drug delivery. Nat. Biotechnol. 36, 707-716 (2018)), are also encompassed for enhancing anti-tumor immunity as described herein.

The invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### IL-10 CAR T Cells Targeting HER2

We first investigated whether CAR T with ectopical IL-10 expression modulates metabolic fitness and improve their antitumor activity. IL-10 HER2 CAR constructs were generated by fusing HER2 CAR and IL-10 gene fragment with 2A self-cleaving peptide into the retroviral vector pMSGV (FIG. 1a). Cell surface expression of HER2 CAR in IL-10 HER2 CAR T was almost equivalent to that in conventional HER2 CAR T cells (FIG. 1b). The IL-10 level produced by IL-10 HER2 CAR was measured by ELISA (FIG. 1c). As IL-10 is known to enhance the proliferation of CD8 T cells upon antigen stimulation (Guo, Y. et al. Metabolic reprogramming of terminally exhausted CD8+ T cells by IL-10 enhances anti-tumor immunity. Nat. Immunol. 22, 746-756 (2021)), we investigated the absolute counts and number of cell divisions of CAR T cells when receiving antigen stimulation. The counts and cell divisions of IL-10 HER2 CAR T cells were significantly higher as compared to those of HER2 CAR T cells when co-cultured with mitomycin treated MC38-HER2 mouse colon cancer cells (FIGS. 1d, e). Enhanced division of IL-10 HER2 CAR T cells were fully attenuated by anti-IL-10 antibody (FIG. 1e), indicating that IL-10 is essential for the improved proliferation of IL-10 HER2 CAR T cells. In a co-culture system of MC38-HER2 cells, the basal oxygen consumption rates (OCR) of IL-10 HER2 CAR T cells were elevated whereas extracellular acidification rate (ECAR) remained unchanged as compared with HER2 CAR T cells (FIGS. 1f-i). On the other hand, IL-10 HER2 CAR T cells showed equivalent basal OCR of HER2 CAR T in the presence of mIL-10. The ratio of OCR to EACR of both HER2 CAR T in the presence of mIL-10 and IL-10 HER2 CAR T were markedly increased (FIG. 1j), suggesting that IL-10 signaling actively promotes oxidative phosphorylation (OXPHOS) of CAR T cell. As a result of metabolic reprograming, the tumor-lytic potential of IL-10 HER2 CAR T was greatly enhanced (FIG. 2a), meanwhile, the antigen specific proliferation capacity, killing efficiency and polyfunctionality of CAR T cells was greatly enhanced in group of IL-10 HER2 CAR T in a coculture environment (at E:T ratios of 0.5:1 for 48 h) that mimicked in vivo scenarios (FIGS. 2b-d). These data support that IL-10 CAR T of the invention is able to reprogram CAR T cells metabolism to promote cell proliferation by increasing OXPHOS, which indicates that IL-10CAR T of the invention could also promote proliferation of tumor infiltrating CAR T cells in the TME through metabolic intervention.

Enhancing OXPHOS or inhibiting glycolytic metabolism in CD8 T cells by various reagents promoted CD8+ T cell proliferation, memory development, and antitumor function in TME (Sukumar, M. et al. Inhibiting glycolytic metabolism enhances CD8+ T cell memory and antitumor function. J. Clin. Invest. 123, 4479-4488 (2013)). Based on the observed metabolic regulation function of the IL-10 HER2 CAR T cells, we next investigated whether in vivo metabolic intervention of CAR T cells can be achieved to enhance the efficacy against solid tumors.

In a therapeutic setting with pre-established MC38-HER2 tumor, adoptive transfer of IL-10 HER2 CAR T cells (3×10⁶) monotherapy with lymphodepletion preconditioning (4Gy) consistently induced complete tumor regression and durable cures in 80% of treated mice (FIGS. 3a-b). In contrast, HER2 CAR T monotherapy had minimal effect on tumor growth inhibition. Adoptive transfer of HER2 CAR T with i.v. administration of mIL-10 only transiently controlled tumor growth but failed to induce tumor regression. Furthermore, all the long-term survivors treated with IL-10 HER2 CAR T cells rejected a rechallenge of MC38-HER2 cells 2 months post cessation of therapy, indicating the induction of antitumor immune memory (FIG. 3c).

### IL-10 CAR T Cells Targeting TRP-1

To test the robustness of IL-10 CAR T cell therapy, we next assessed whether a poorly immunogenic and highly aggressive mouse B16F10 melanoma model could also be controlled. We next generated IL-10 CAR T cells targeting to TRP-1, and expression of CAR was confirmed by flow cytometry (FIGS. 4a, b). In vitro cell proliferation and antitumor activity of IL-10 TRP-1 CAR T cells were superior to those of conventional TRP-1 CAR T cells (FIGS. 4c, d). Mice bearing B16F10 mouse melanoma were lymphodepleted by irradiation (4Gy) before the CAR T cell transfer. IL-10 TRP-1 CAR T led to remarkable tumor regression and eventually elimination in most tumor bearing mice, while TRP-1 CAR T showed only transient tumor growth inhibition without durable therapeutic effect (FIG. 4e). In addition, 60% of mice treated with IL-10 TRP-1 CAR T therapy exhibited long-term survival (FIG. 4f).

### IL-10 CAR T Cells Targeting EGFRvIII

We next further extended the IL-10 CAR T of the intervention to a highly aggressive and metastatic 4T1-Luc-EGFRvIII (stably transfected with EGFRvIII and luciferase (Luc)) mouse mammary carcinoma model. We prepared IL-10 CAR T cells targeting EGFRvIII. Expression of CAR and IL-10 production were confirmed by flow cytometry and ELISA, respectively (FIGS. 5a-c). In vitro cell expansion and antitumor activity of IL-10 EGFRvIII CAR T cells were significantly improved as compared with those of EGFRvIII CAR T cells (FIGS. 5d, e). BALB/c mice were i.v. injected with 4T1-Luc-EGFRvIII tumor cells (5×10⁵) to develop lung metastases. Transfer of EGFRvIII CAR T cells (3×10⁶) with or w/o mIL-10 administration to mice resulted in transient tumor inhibition (FIG. 5f). Notably, IL-10 EGFRvIII CAR T cells completely eradicated tumors and led to durable cures in 100% of treated mice (FIG. 5g). Consistent with the potent antitumor efficacy, IL-10 EGFRvIII CAR T cells induced a remarkably high density of CAR T cells in circulation (FIG. 5h).

These results indicated IL-10 HER2 CAR T exhibited improved in vivo expansion, augmented functionality, eventually contributing to superior efficacy of IL-10 expressing CAR T cells.

### IL-10 TCR T (Pmel) Cells

To further extend the IL-10 expressing T cell strategy to tumor specific T cell receptor (TCR) transgenic T cells (TCR T) against highly aggressive B16F10 mouse melanoma tumor model, we prepared IL-10 expressing Pmel T cells (IL-10 Pmel T) similarly as previous description and the expression of IL-10 production were confirmed by ELISA (FIG. 6a). Mice bearing B16F10 mouse melanoma were received adoptive transfer of PBS control, Pmel T or IL-10 Pmel T cells. IL-10 Pmel T cells led to remarkable tumor regression in most tumor bearing mice, while Pmel T cells showed only modest tumor growth inhibition (FIG. 6b-e). In addition, the mice treated with IL-10 expressing Pmel T cells exhibited improved survival compared to those treated with Pmel T cells (FIG. 6f). Similarly, this IL-10 expressing TCR T cell strategy could also be used for enhanced efficacy of adoptive transfer therapy of TILs against solid tumors.

### Human IL-10 CAR T Cells Targeting CD19

At last, we extended this IL-10 expressing CAR T strategy to human CAR T. IL-10 CD19 CAR constructs were generated by fusing CD19 CAR and human IL-10 gene fragment with 2A self-cleaving peptide into the lentiviral vector (FIG. 7a). Cell surface expression of CD19 CAR in IL-10 CD19 CAR T was slightly higher than that in conventional CD19 CAR T cells (FIG. 7b). The IL-10 level produced by IL-10 CD19 CAR was measured by ELISA (FIG. 7c). Accordingly, the tumor-lytic potential of IL-10 CD19 CAR T was also enhanced (FIG. 7d), consistent with results we observed in mouse CAR T. Furthermore, in immune-deficient NSG mice bearing established PANC1-CD19 human pancreatic tumors, all mice treated with IL-10-expressing CD19 hCAR T cells exhibited complete remission with no recurrence (FIG. 7e), suggesting that IL-10-expressing human CAR T cells had superior anti-tumor capacity against solid tumors in a xenograft model.

### IL-10-Expressing CAR-T Cells Sustain Mitochondrial Fitness

Impaired mitochondrial fitness has been shown to reinforce T cell exhaustion. Intriguingly, we found IL-10 expression sustained the mitochondria fitness in tumor-infiltrating CAR-T cells with substantially reduced frequency of dysfunctional mitochondria in IL-10 HER2 CAR-T cells (4.8%) as compared to HER2 CAR-T cells alone (27.4%) or HER2 CAR-T cells combined with exogenous IL-10 (21.7%) (FIG. 8a). IL-10 expression also increased the ratio of MDR to MG in IL-10 HER2 CAR-T cells (FIG. 8b). EM imaging analysis of mitochondrial ultrastructure provided additional evidence of enriched mitochondria with tubular shape, well-structured cristae, increased cristae numbers, and enlarged length of cristae per mitochondrion in tumor-infiltrating IL-10 HER2 CAR-T cells as compared to conventional HER2 CAR-T cells (FIGS. 8c-f).

### IL-10 Expression in CAR-T Cells Induces Durable Anti-Tumor Immunity and Promotes Stemness

To investigate whether IL-10-expressing CAR-T cells developed anti-tumor immune memory, we rechallenged the surviving mice 3 months post adoptive CAR-T cell transfer. Impressively, 100% of long-term survivors treated with IL-10 HER2 CAR-T or IL-10 TRP-1 CAR-T cells rejected a second challenge of the original tumor cells (FIGS. 9a-c). The robust immune memory response motivated us to examine the memory phenotypes of IL-10-expressing CAR-T cells in the lymphoid tissues and the circulation. Flow cytometry analysis of CAR-T cells (initially transferred CAR-T cells were all CD44hi) 12 days post treatment showed that IL-10 HER2 CAR T cells enriched a population with Tscm phenotype (defined by CD62LhiCD44lo and stem cell antigen-1 (Sca-1)+CD122+) in spleen and peripheral blood (FIGS. 10a and b). IL-10 HER2 CAR-T cells in the spleen showed ˜3.2-fold higher frequency of CD62LhiCD44lo T cells compared to HER2 CAR-T cells alone, among which the majority (˜71.2%) were Sca-1+CD122+ Tscm (FIGS. 10a and b). In addition, IL-10 HER2 CAR-T cells exhibited substantially increased expression of Sca-1 compared to HER2 CAR-T cells alone or HER2 CAR-T cells plus exogenous IL-10 (FIG. 10c). This finding was further confirmed by the observation that IL-10 HER2 CAR T cells were composed of ˜3.7- and 2.6-fold higher proportion of IL-7Ra+KLRG1-long-lived memory precursor T cells than HER2 CAR-T cells in spleen and blood, respectively (FIGS. 10d and e). Additionally, we observed that compared to CD19 hCAR-T cells, treatment of IL-10 CD19 hCAR-T cells appeared enriched with Tscm. Altogether, these results implicate IL-10 signaling may induce the formation of mouse and human Tscm CAR-T cells contributing to long-term anti-tumor immunity.

Sequences
human IL-10
Amino acid sequence
SEQ ID NO: 1
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCENKSKAVEQVKNAFNKLQEKGIYKAMSEFDIFINYI
EAYMTMKIRN
HER2 scFV
Amino acid sequence
SEQ ID NO: 2
DYKDIVMTQSPSSLSASVGDRVTITCRASQDVNTAVAWYQQKPGKAPKLL
IYSASFLYSGVPSRFSGSRSGTDFTLTISSLQPEDFATYYCQQHYTTPPT
FGQGTKVELKRATPSHNSHQVPSAGGPTANSGEVKLVESGGGLVQPGGSL
RLSCATSGFNIKDTYIHWVRQAPGKGLEWVARIYPTNGYTRYADSVKGRF
TISADTSKNTAYLQMNSLRAEDTAVYYCSRWGGDGFYAMDYWGQGTTVTV
SS
TRP-1 scFV
Amino acid sequence
SEQ ID NO: 3
EVQLQQSGAELVRPGALVKLSCKTSGFNIKDYFLHWVRQRPDQGLEWIGW
INPDNGNTVYDPKFQGTASLTADTSSNTVYLQLSGLTSEDTAVYFCTRRD
YTYEKAALDYWGQGASVIVSSGGGGSGGGGSGGGGSDIQMSQSPASLSAS
VGETVTITCRASGNIYNYLAWYQQKQGKSPHLLVYDAKTLADGVPSRFSG
SGSGTQYSLKISSLQTEDSGNYYCQHFWSLPFTFGSGTKLEIKRA
EGFRvIII ScFV
Amino acid sequence
SEQ ID NO: 4
EVQVLESGGGLVQPGGSLRLSCAASGFTFSSYAMSWVRQAPGKGLEWVSA
ISGSGGSTNYADSVKGRFTISRDNSKNTLYLQMNSLRAEDTAVYYCAGSS
GWSEYWGQGTLVTVSSGGGGSGGGGSGGGGSDIQMTQSPSSLSASVGDRV
TITCRASQGIRNNLAWYQQKPGKAPKRLIYAASNLQSGVPSRFTGSGSGT
EFTLIVSSLQPEDFATYYCLQHHSYPLTSGGGTKVEIK
CD19 ScFv
Amino acid sequence
SEQ ID NO: 5
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS
CD8 transmembrane domain and hinge
Amino acid sequence
SEQ ID NO: 6
TTTKPVLRTPSPVHPTGTSQPQRPEDCRPRGSVKGTGLDFACDIYIWAPL
AGICVALLLSLIITLICYHRSR
CD3ζ
Amino acid sequence
SEQ ID NO: 7
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

1. An immune cell expressing an interleukin-10 or a fragment or a variant thereof, said immune cell comprising one or more recombinant constructs, wherein at least one recombinant construct encodes an interleukin-10 or a fragment or a variant thereof.

2. The immune cell of claim 1, wherein a second recombinant construct encodes a chimeric antigen receptor (CAR), a T cell receptor (TCR) or any other synthetic tumor targeting motif.

3. The immune cell of claim 1, wherein the immune cell is a T cell, chimeric antigen receptor (CAR)-T cell, T cell receptor (TCR)-transgenic T cell, tumor infiltrating lymphocyte (TIL), NK cell, NK-T cell, CAR-NK cell, CAR-NKT cell, TCR-transgenic NK cell, TCR-transgenic NK-T cell, dendritic cell, macrophage, CAR-macrophage, or any synthetic tumor specific immune cells.

4. The immune cell of claim 1, wherein the construct encoding an interleukin-10, a fragment or a variant thereof comprises or encodes a sequence of SEQ ID NO:1 or a fragment or variant thereof.

5. The immune cell of claim 1, wherein the recombinant construct encoding an interleukin-10, a fragment or a variant thereof is linked to the second recombinant construct encoding a CAR, a TCR, or any other synthetic tumor targeting motif.

6. The immune cell of claim 5, wherein the recombinant construct encoding an interleukin-10, a fragment or a variant thereof is linked to the second recombinant construct encoding a CAR, a TCR, or any other synthetic tumor targeting motif; via a sequence encoding a self-cleaving peptide.

7. The immune cell of claim 5, wherein the second recombinant construct encoding the CAR, contains an extracellular antigen recognition domain of a single-chain Fragment variant (scFv) derived from an antibody.

8. The immune cell of claim 5, wherein the second recombinant construct encoding the CAR contains a nucleic acid encoding a polypeptide of a transmembrane region.

9. The immune cell of claim 5, wherein the second recombinant construct encoding the CAR contains a nucleic acid encoding a polypeptide of an intracellular T cell activation domain of CD3ζ.

10. The immune cell claim 5, wherein the second recombinant construct encoding the CAR contains a 4-1BB or CD28 or a combination of 4-1BB and CD28 intracellular region.

11. The immune cell of claim 7, wherein
i) the extracellular antigen recognition domain of the scFv derived from an antibody recognizes an antigen selected from the group consisting of c-MET, CD7, CD19, CD20, CD22, CD38, CD123, CD133, CD171, CD70, BCMA, CEA, EGFR-VIII, EpCAM, EphA2, FAP, GD2, GPC3, HER2, IL-13Ra2, Mesothelin, MUC1, PSCA, PSMA, ROR1, VEGFR2, and Claudin 18.2; or
ii) the TCR recognizes an antigen selected from the group consisting of gp100, NY-ESO-1, MAGE-A3, TRP-1, and a combination of one or more thereof.

12. The immune cell of claim 1, wherein the interleukin-10, a fragment or a variant thereof, is secreted or membrane bound by the immune cell.

13. The immune cell of claim 1, wherein the construct encoding an interleukin-10, a fragment or a variant thereof is comprised within or without a sequence encoding a Fc, HSA, or antibody fusion protein.

14. (canceled)

15. (canceled)

16. The immune cell of claim 1, wherein the immune cell is autologous, allogeneic, or heterologous.

17. The immune cell of claim 7, wherein the extracellular antigen recognition domain of the scFv derived from an antibody recognizes an antigen selected from the group consisting of HER2, TRP-1, EGFRvIII, and CD19.

18. The immune cell of claim 17, wherein the amino acid sequence of the scFv recognizing HER2 comprises SEQ ID NO: 2 or a fragment or a variant thereof.

19. The immune cell of claim 17, wherein the amino acid sequence of the scFv recognizing TRP-1 comprises SEQ ID NO: 3 or a fragment or a variant thereof.

20. The immune cell of claim 17, wherein the amino acid sequence of the scFv recognizing EGFRvIII comprises SEQ ID NO: 4 or a fragment or a variant thereof.

21. The immune cell of claim 17, wherein the amino acid sequence of the scFv recognizing CD19 comprises SEQ ID NO: 5 or a fragment or a variant thereof.

22. The immune cell of claim 8, wherein the amino acid sequence of the transmembrane domain and hinge comprises SEQ ID NO: 6 or a fragment or a variant thereof.

23. The immune cell of claim 9, wherein the amino acid sequence of the polypeptide of the intracellular T cell activation domain of CD3ζ comprises SEQ ID NO: 7 or a fragment or a variant thereof.

24. A pharmaceutical composition comprising a therapeutically effective amount of:
i) an immune cell expressing an interleukin-10 or a fragment or a variant thereof, said immune cell comprising one or more recombinant constructs, wherein at least one recombinant construct encodes an interleukin-10 or fragment or a variant thereof,
ii) a nucleic acid encoding the one or more recombinant constructs; of i); and/or
iii) a plasmid or a vector comprising the nucleic acid sequence of ii).

25. The pharmaceutical composition of claim 24, further comprising at least one additional therapeutic agent or therapy.

26. The pharmaceutical composition of claim 25, wherein the at least one additional therapeutic agent or therapy is an anticancer agent or anticancer therapy, and/or an anti-inflammatory agent.

27. The pharmaceutical composition of claim 26, wherein the anti-cancer therapy comprises radiotherapy, chemotherapy, an immune checkpoint inhibitor, immunotherapy, and-hormone therapy, or a combination of one of more thereof.

28. The pharmaceutical composition of claim 27, wherein the immune checkpoint inhibitor is selected from the group consisting of a PD-1 inhibitor, a PD-L1 inhibitor, a CTLA-4 inhibitor, and a combination of one of more thereof.

29. A method of treating cancer in a subject in need thereof, the method comprising administering the pharmaceutical composition of claim 24 to the subject.

30. A method of treating a cancer in a subject in need thereof, the method comprising:
(i) genetically engineering native T cells from the subject with at least one recombinant construct encoding an interleukin-10; or a fragment or a variant thereof and with a second recombinant construct encoding a chimeric antigen receptor (CAR), a T cell receptor (TCR) or any other synthetic tumor targeting motif or antigen to produce engineered T cells,
(ii) expanding the engineered cells ex vivo into a larger population of engineered T cells, and
(iii) introducing the expanded engineered T cells into the subject.

31. A method of enhancing antitumor activity in a subject in need thereof the method comprising:
(i) genetically engineering said-native T cells from subject with at least one recombinant construct encoding an interleukin-10; or a fragment or a variant thereof and with a second recombinant construct encoding a chimeric antigen receptor (CAR), a T cell receptor (TCR) or any other synthetic tumor targeting motif or antigen to produce engineered T cells,
(ii) expanding the engineered T cells ex vivo into a larger population of engineered T cells, and
(iii) introducing into the expanded engineered T cells into the subject.

32. The method of claim 29, wherein the cancer is selected from the group comprising lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer, brain cancer and skin cancer, in particular melanoma, or a combination of one or more thereof.


SEQUENCE LISTING (7 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 252 aa, unidentified
  REGION 1..252: note = Artificial sequence
DYKDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSS

SEQ ID NO: 3 - protein, 245 aa, unidentified
  REGION 1..245: note = Artificial sequence
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSSGGGGSGGGGSGGGGSDIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRA

SEQ ID NO: 4 - protein, 238 aa, unidentified
  REGION 1..238: note = Artificial sequence
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSGGGGSGGGGSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIK

SEQ ID NO: 5 - protein, 242 aa, unidentified
  REGION 1..242: note = Artificial sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 6 - protein, 72 aa, unidentified
  REGION 1..72: note = Artificial sequence
TTTKPVLRTPSPVHPTGTSQPQRPEDCRPRGSVKGTGLDFACDIYIWAPLAGICVALLLSLIITLICYHRSR

SEQ ID NO: 7 - protein, 113 aa, unidentified
  REGION 1..113: note = Artificial sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR